(19) United States
(12) Patent Application Publication
(10) Pub. No.: US 2018/0154103 A1
(43) Pub. Date: Jun. 7, 2018

(54) VAPORIZER APPARATUS FOR COMPRESSED TABLET AND LOOSE FILL PLANT SOURCE MATERIALS

(71) Applicant: Compressed Perforated Puck Technologies Inc., Edmonton (CA)

(72) Inventor: David DAVIS, Edmonton (CA)

(73) Assignee: Compressed Perforated Puck Technologies Inc., Edmonton, AB (CA)

(21) Appl. No.: 15/576,480

(22) PCT Filed: May 20, 2016
(86) PCT No.: PCT/CA2016/000151

§ 371 Date: Nov. 22, 2017

Related U.S. Application Data
(60) Provisional application No. 62/165,738, filed on May 22, 2015.
(60) Provisional application No. 62/195,123, filed on Jul. 21, 2015.

(51) Int. Cl. A61M 16/10; A61M 16/00; A61M 16/20; A61M 15/00; A24F 47/00; A61J 3/10; H05B 6/06; H05B 6/10

(52) U.S. Cl. CPC A61M 16/1075; A61M 2209/06; A61M 16/107; A61M 16/209; A61M 15/002; A61M 15/0035; A61M 15/0066; A24F 47/008; A61J 3/10; H05B 6/06; H05B 6/108; A61M 2205/07; A61M 2205/14; A61M 2205/3334; A61M 2205/3368; A61M 2205/3584; A61M 2205/3606; A61M 2205/362; A61M 2205/502; A61M 2205/583; A61M 2205/6072; A61M 2205/7545; A61M 2205/8206; A61M 16/0066

(57) ABSTRACT

There is disclosed a vaporizer apparatus for a compressed tablet formed from a plant source material containing medicinal ingredients of therapeutic efficacy. In an embodiment, the apparatus includes: a holder for a compressed tablet; a microprocessor; a controlled air flow; and a controlled heat source; wherein the microprocessor is adapted to control the air flow and the heat source to vaporize the compressed tablet received in the compressed tablet holder at a desired rate. In another embodiment, the vaporizer apparatus includes a carousel for receiving a disc cartridge containing packaged compressed tablets. In still another embodiment, the vaporizer apparatus is adapted to recognize a type of compressed tablet placed into the holder, and to control an air flow and a heat source based on selected therapeutic compounds desired to be released from the recognized type of compressed tablet.

### FIELD

The present disclosure relates to vaporizers, and more generally to vaporizers for vaporizing plant source materials to release medicinal ingredients.

### BACKGROUND

Many medicinal ingredients of therapeutic efficacy for various conditions or ailments may be found in plant sources. In some cases, delivering these medicinal ingredients from the plant source may involve subjecting the plant source material to combustion in order to release the active ingredients in the plant source material for inhalation. While conventional methods such as lighting and inhaling the resulting smoke of the burning plant source material may provide effective delivery of the active ingredients, there may also be adverse side effects resulting from formation of toxic compounds in the gaseous and airborne particles in the smoke formed from combustion. Such toxins in the smoke may include neurotoxins which may be poisonous or destructive to nerve tissue of the individuals inhaling them. There may also be other toxins which have the potential to damage the respiratory system or cardiovascular system of these individuals when this delivery method is used repeatedly over a long period of time.

Prior art vaporizers come in all shapes and sizes, varying in quality and functionality. The majority of vaporizers contain a small chamber and a heat source, normally an electric heating element. This heating element heats air which is passed through the chamber containing the plant source material using a small fan or by inhalation, depending on how advanced the vaporizer is. Through conduction and/or convection, a vapor loaded with the active ingredients of the plant source material is obtained.

However, prior art vaporizers are limited in their ability to control the rate of vaporization and the effectively release of different therapeutic compounds in plant source materials in part due to the problems in dealing with loose plant source materials used in the chamber.

What is therefore needed is an improved technological solution that overcomes at least some of these limitations.

### SUMMARY

The present disclosure relates to a vaporizer apparatus which vaporizes plant source material containing medicinal ingredients of therapeutic efficacy.

In an embodiment, the present vaporizer is adapted to vaporize compressed tablets formed from plant source materials. The compressed tablets are received in a heating chamber with a controlled heating element to significantly increase control over the vaporization of certain constituent therapeutic compounds at desired rates of vaporization in order to optimize efficacy of the therapeutic compounds and maintain dosage consistency from one therapeutic session to the next.

In another embodiment, the vaporizer apparatus is a table top model having a carousel for receiving a disc cartridge containing a plurality of compressed tablets. The carousel is adapted to rotate to position a compressed tablet within a heating chamber in order to vaporize it, and once the tablet is spent, to advance the disc cartridge to the next compressed tablet in the cartridge.

In another embodiment, the vaporizer apparatus includes a compressed tablet type detector configured to recognize different types of compressed tablets based on one or more distinguishing features. Such a distinguishing feature may include, for example, the shape of the tablet or a pattern of features such as a plurality of holes or ribbed edges detectable by sensors, or a machine readable label such as a bar code, QR code, or an RFID tag provided on the compressed tablet or on packaging for the compressed tablet.

In another embodiment, the vaporizer apparatus may also be used to vaporize plant source material processed as loose fill plant source material contained in a mesh container or basket. The vaporizer apparatus may include a detector configured to recognize different types of loose fill plant source material based on a machine readable label such as a bar code, QR code, or an RFID tag provided on the packaging or a mesh container or basket for the loose fill plant source material.

In another embodiment, the vaporizer apparatus includes a microcontroller adapted to control a temperature profile designed to release therapeutic compounds from the compressed tablet or loose fill plant source material at a desired rate over a set period of time.

In another embodiment, the microcontroller may vary the temperature profile over a set period of time in order to target specific therapeutic compounds, or a blend of different therapeutic compounds selected to alleviate specific conditions or ailments.

In another embodiment, the microcontroller may vary the rate of air flow through the heating chamber in order to control the amount of vapor delivered to the user.

In another embodiment, one or more filters may be used to reduce any noxious substances that may be contained in the vapor.

In this respect, before explaining at least one embodiment of the system and method of the present disclosure in detail, it is to be understood that the present system and method is not limited in its application to the details of construction and to the arrangements of the components set forth in the following description or illustrated in the drawings. The present system and method is capable of other embodiments and of being practiced and carried out in various ways. Also, it is to be understood that the phraseology and terminology employed herein are for the purpose of description and should not be regarded as limiting.

### BRIEF DESCRIPTION OF THE DRAWINGS

FIG. 1A shows a schematic block diagram of a vaporizer in accordance with an illustrative embodiment.

FIG. 1B shows a schematic air flow pattern in accordance with an illustrative embodiment.

FIGS. 2A to 2D show an illustrative compressed vaporizer tablet and optional blister packaging which may be received in a vaporizer in accordance with an embodiment.

FIGS. 3A to 3D show illustrative views of an air heater assembly in accordance with an embodiment.

FIGS. 4A to 4F show illustrative views of an enclosure fitting in accordance with an embodiment.

FIGS. 5A to 5F show illustrative views of a heat sink in accordance with an embodiment.

FIGS. 6A to 6F show illustrative views of an alternative heat sink in accordance with an embodiment.

FIGS. 7A to 7C show illustrative views of a hose barb in accordance with an embodiment.

FIGS. 8A to 8D show illustrative views of a lower spring holder in accordance with an embodiment.

FIGS. 9A to 9C show illustrative views of an alternative embodiment of the vaporizer including a relief valve for gassing-off selected compounds.

FIG. 10 shows an illustrative mesh basket and screen disc for containing loose fill plant source material in accordance with an embodiment.

FIG. 11 shows a schematic air flow pattern in accordance with another embodiment.

FIGS. 12A to 12D show illustrative views of an air heater assembly in accordance with another embodiment.

FIG. 13 shows a schematic block diagram of a vaporizer in accordance with another illustrative embodiment.

FIG. 14A shows an illustrative perspective view of a table top embodiment of the vaporizer of FIG. 13 having a carousel adapted to receive a disc cartridge of compressed vaporizer tablets.

FIG. 14B shows a partial see-through perspective view of various components in the table top embodiment of FIG. 14A.

FIG. 15 shows a schematic top view of various components within the table top vaporizer of FIGS. 14A and 14B.

FIG. 16 shows a front view of an illustrative disc cartridge of compressed vaporizer tablets in accordance with an embodiment.

FIG. 17 shows a schematic side view of various components within the table top vaporizer of FIGS. 14A and 14B.

FIG. 18 shows a schematic diagram of LED indicators which may be provided on the table top vaporizer of FIGS. 14A and 14B.

FIG. 19 shows a schematic diagram of a magnetic heater in accordance with an embodiment.

FIG. 20 shows a schematic block diagram of a basic induction heating subsystem in accordance with an embodiment.

FIG. 21 shows a schematic block diagram of the induction heating subsystem of FIG. 20 heating a flow of air from an air pump to heat a compressed vaporizer tablet in accordance with an embodiment.

FIG. 22 shows a schematic block diagram of an alternative heating subsystem which utilizes a plug style heater rather than an induction heating design

FIG. 23 shows a schematic block diagram of a control subsystem for controlling multivariable processes in the system accordance with an embodiment.

FIG. 24 shows a schematic block diagram of the control subsystems of FIG. 23 controlling the various subsystems of FIG. 21.

### DETAILED DESCRIPTION

As noted above, the present disclosure relates to a vaporizer apparatus which vaporizes plant source material containing medicinal ingredients of therapeutic efficacy.

Prior art vaporizers are limited in their ability to control the rate of vaporization and the effectively release of different therapeutic compounds in plant source materials, in part due to the problems in dealing with loose plant source materials as used in the chamber of prior art devices.

Some plant source materials may contain hundreds of constituent parts. For example, in the cannabis plant, there are 483 identifiable chemical constituents known to exist, at least 85 of which are different cannabinoids have been isolated from the plant. These constituent parts have different vaporization points. For example, the aromatic terpenoids begin to vaporize at 126.0° C. (258.8° F.), while the more bioactive compounds such as tetrahydrocannabinol (THC), cannabidiol (CBD) and cannabinol (CBN) do not vaporize until near their respective boiling points: THC 157° C. (315° F.); CBD 160 180° C. (320° F.-356° F.)); and CBN 185° C. (365° F.).

Various factors that may affect the rate of vaporization include specimen density; weight, content of water and essential oils; consistency of material in the filling chamber; storage time of the vapor; and the inhalation method used (breathing technique). Not all these have been scientifically tested. However, research at Leiden University using vaporizers found the delivery efficiency highest at around 226° C. (439° F.), falling to about half efficiency at 150° C. (302° F.) to 180° C. (356° F.) depending on material. The purest preparations produced the highest efficiencies, about 56% for pure THC versus 29% for plant material (female flower tops) with 12% THCA content. Besides THC, several other cannabinoids as well as a range of other plant components including terpenoids were detected in the plant material. Using pure THC in the vaporizer, no degradation products (delta-8-THC (D8-THC), cannabinol (CBN), or unknown compounds) were detected by HPLC analysis. The longer vapor is stored, the more THC is lost as it condenses on the surface of the vaporizer or the balloon. This loss may be negligible over a few minutes but may exceed 50% after 90 minutes. The Leiden University study found that as much as 30%-40% of inhaled THC was not absorbed by the lungs but simply exhaled. However, they did not find large individual differences in the amounts exhaled.

To the inventor's knowledge, all existing vaporizers require the plant source material to be loosely packed. In contrast, the present vaporizer is adapted to utilize compressed tablets received in a heating chamber to significantly increase control over the vaporization of certain constituent compounds, at desired rates of vaporization, in order to optimize the inhalation and efficacy of the therapeutic compounds and maintain dosage consistency from one session to the next.

In another embodiment, the vaporizer apparatus includes a carousel, which is adapted to utilize compressed tablets packaged in a disc cartridge. The carousel is rotatable to place another compressed tablet into position for vaporization in the heating chamber, after the current tablet is spent.

In another embodiment, the vaporizer apparatus is able to use processed and measured loose fill plant source material contained in a mesh container or basket to control dosage.

In another embodiment, by setting specific temperatures for a given compressed tablet type, or for a processed and measured loose fill plant source material contained in a mesh container or basket, the vaporizer apparatus enables therapeutic compounds in the compressed tablet or loose fill plant source material to be vaporized in dependence upon their evaporating temperature.

In another embodiment, precision temperature sensors are adapted to monitor the air exiting the heating chamber. Air speed and heating element temperature, controlled through the microprocessor, allows for temperature stability to within a few degrees of the intended temperature setting.

In another embodiment, the present vaporizer apparatus includes a microcontroller adapted to control a temperature profile designed to release therapeutic compounds from the compressed tablet at a desired rate over a set period of time.

In another embodiment, the microcontroller of the present vaporizer apparatus may vary the temperature profile over a set period of time in order to release each therapeutic compound of a blend of different therapeutic compounds selected to alleviate specific conditions or ailments.

In another embodiment, the microcontroller may vary the rate of air flow through the heating chamber in order to control the amount of vapor delivered to the user.

In another embodiment, rather than relying on a fan, the present vaporizer apparatus incorporates an air pump which is fed through a precision flow meter before entering an electric heating chamber. The flow meter measures the amount of air passing over the heating element and into a chamber holding the compressed tablet, before being delivered to the user.

In another embodiment, as the vaporizer apparatus monitors the total amount of heated air going through the compressed tablet, and knows when the compressed tablet was placed in the unit (through addition sensors), it can notify the user as to when to replace the compressed tablet or container for the loose fill plant source material.

Advantageously, utilizing a precision air flow meter together with a controlled temperature profile for a given compressed tablet type or container of processed and measured loose fill plant source material contained in a mesh container or basket, metered doses can then be consistently administered to the user from one therapeutic session to the next.

Various illustrative examples of the vaporizer apparatus will now be described with reference to the drawings.

A vaporizer or vaporiser is a device used to vaporize the active ingredients of plant source material, commonly cannabis, tobacco, or other herbs or blends for the purpose of inhalation. However, they can be used with pure chemicals when mixed with plant material (e.g. tobacco-free nicotine).

Vaporizers come in all shapes and sizes, varying in quality and functionality. The majority of vaporizers contain a small chamber and a heat source, normally an electric heating element. This heating element heats up the air, which is passed through the chamber containing the plant material using a small fan, or by inhalation depending on how advanced the vaporizer is. Through the powers of convection, you gain a vapor loaded with the active ingredients of the plant material.

Vaporizers may contain various forms of extraction chambers including straight bore, venturi, or sequential venturi, and are made of materials such as metal or glass. The extracted vapor may be collected in an inflatable bag, or inhaled directly through a hose or pipe. With no combustion happening when used properly and cooler temperatures, a significantly better efficiency in extracting the ingredients can be obtained. Hence, the irritating and harmful effects of smoking are heavily reduced, as is second-hand smoke.

Vaporizers may also be used to inhale cannabis for therapeutic purposes. As heated air heats up the dried plant source material, the therapeutic compounds within the plant source material melt into a vapor and are carried through the vaporizer. Depending on the vaporizer, this can be inhaled directly or stored in a balloon like bag for gradual consumption. Of the studies about vaporizing plant source material, few have addressed the quality of the vapor extracted and delivered; instead, studies usually focus on the mode of usage of the vaporizers. There are 483 identifiable chemical constituents known to exist in the cannabis plant, and at least 85 different cannabinoids have been isolated from the plant. The aromatic terpenoids begin to vaporize at 126.0° C. (258.8° F.), but the more bio-active tetrahydrocannabinol (THC), cannabidiol (CBD) and cannabinol (CBN) do not vaporize until near their respective boiling points: THC 157° C. (315° F.), CBD 160-180° C. (320° F-356° F),^{[17]} and CBN 185° C. (365° F.).

Studies have shown that vaporizing cannabis plant source materials exposes the user to lower levels of harmful substances than smoking cannabis. These findings are important for it is estimated that 10-20 percent of patients with chronic pain, multiple sclerosis, epilepsy, and HIV/AIDS have admitted to smoking cannabis for therapeutic purposes. For patients, a study found that smoking cannabis sativa reduced daily pain by 34%, a statistically significant amount.

In a study published in the Journal of Psychopharmacology in May 2008, it was stated that vaporizers were a “suitable method for the administration of THC.” A 2007 study by University of California, San Francisco, published in the Journal of the American Academy of Neurology, founded that “there was virtually no exposure to harmful combustion products using the vaporizing device.” A 2006 study performed by researchers at Leiden University found that vaporizers were “safe and effective cannabinoid delivery system(s).” The study stated that the amount of THC delivered by vaporizers were equivalent to the amount delivered by smoking. Because of those studies and other studies, vaporizers are medically sound devices for delivering THC.

The proposed factors affecting output include:

Temperature

Specimen density

Weight, content of water and essential oils

Consistency of material in the filling chamber

Storage time of the vapor

Inhalation method (breathing technique)

Not all those have been scientifically tested. Research using vaporizers found the delivery efficiency highest at around 226° C. (439° F.), falling to about half efficiency at 150° C. (302° F.) to 180° C. (356° F.) degrees depending on material. The purest preparations produced the highest efficiencies, about 56% for pure THC versus 29% for plant material (female flower tops) with 12% THCA content. Besides THC, several other cannabinoids as well as a range of other plant components including terpenoids were detected in the plant material. Using pure THC in the vaporizer, no degradation products (delta-8-THC (D8-THC), cannabinol (CBN), or unknown compounds) were detected by HPLC analysis. The longer vapor is stored, the more THC is lost as it condenses on the surface of the vaporizer or the balloon. This loss may be negligible over a few minutes but may exceed 50% after 90 minutes. The Leiden University study found that as much as 30%-40% of inhaled THC was not absorbed by the lungs but simply exhaled. However, they did not find large individual differences in the amounts exhaled.

### Illustrative Vaporizer Schematic

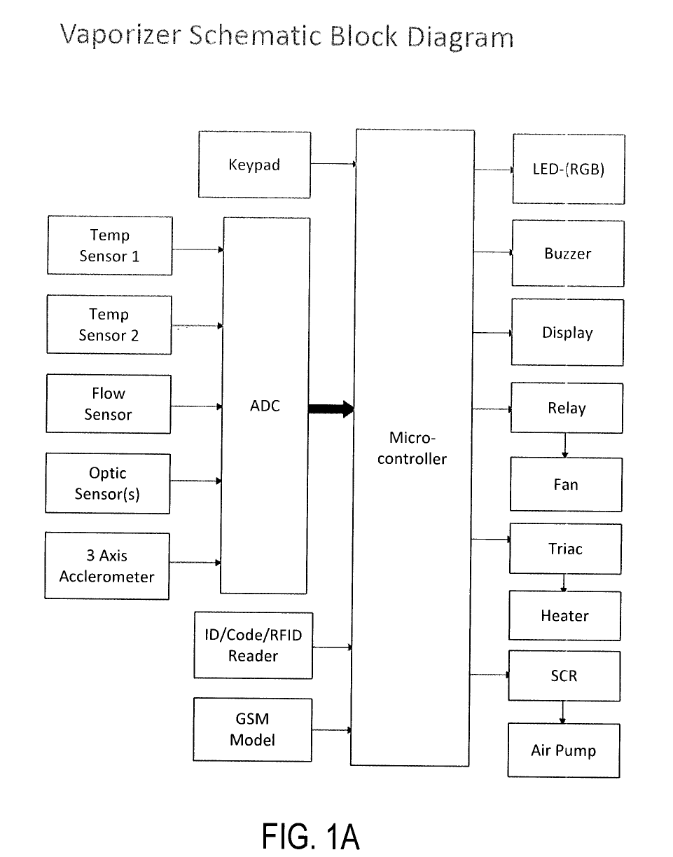

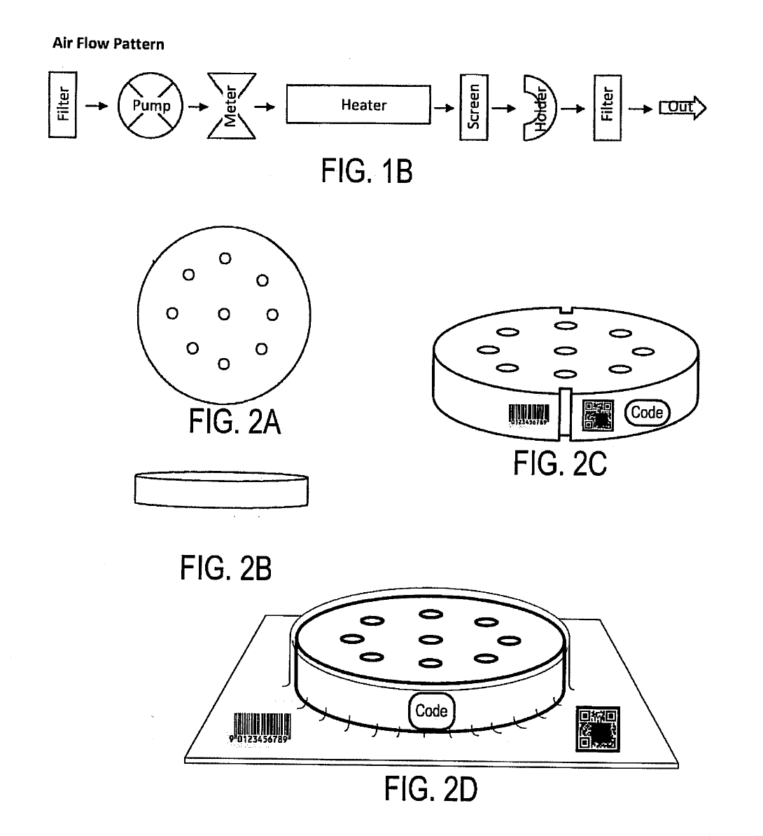

Referring to FIG. 1A, shown is a schematic block diagram of a vaporizer in accordance with an illustrative embodiment. In this illustrative example, temperature sensors monitor the temperature of a heat sink. Two sensors provide redundancy in case one should fail.

In an embodiment, an axis accelerometer is used to detect if the vaporizer is not in the upright position or has been dropped—shutting off the vaporizer. A flow sensor measures the flow rate of air coming from the pump. This will determine dosage amounts and detect if there is a flow blockage resulting in an error message. One or more optic sensors are electronic detectors that convert light, or a change in light, into an electronic signal. The one or more optic sensors will detect when the enclosure fitting has been removed or is in place. A second sensor may indicate when the lid to the vaporizer has been lifted on automatically turning on the vaporizer.

A microcontroller containing a processor core, memory, and programmable input/output peripherals may also be built into the vaporizer to control its various functions. A keypad allows inputs of user choices into the vaporizer. A color liquid crystal display presents relevant information to the user, such as: date/time; set temperature; actual temperature; keypad button definitions; dialogue, symbols and pictures.

In another embodiment, an Analog-to-Digital Converter (ADC) converts a continuous physical quantity (usually voltage) to a digital number that represents the quantity's amplitude. An ADC may also provide an isolated measurement such as an electronic device that converts an input analog voltage or current to a digital number proportional to the magnitude of the voltage or current.

In another embodiment, an ID/Code/RFID reader is provided for reading a machine readable code, such as a bar code, QR code, or another type of code such as an RFID (Radio-frequency identification) which may contain optically or electronically readable information. In the present illustrative embodiment, this machine reader is able to read the prescription information contained in the codes when the compressed tablets or packaging (e.g. blister packs) of the compressed pucks/tablets are brought within reading distance of the vaporizer. For example, and RFID tag may be readable when it is in close proximity (e.g. within six inches) of the vaporizer.

In an embodiment, a built-in cellular modem in the vaporizer periodically connects wirelessly to a database and uploads user vaporizing data. Programming updates may also occur via this wireless connection.

An LED may be used to provide indication of status or mode. For example, using an RGB diode, red, green, and blue light may be used in any combination to indicate the status of the vaporizer. By way of example, and not as a limitation, the light combinations could be: Red—Power on; Green—Up to temperature, ready to dose; Blue—Gassing off; Flashing Red—Error-over temperature, unit dropped, plugged air pathway; Flashing Blue—Replace compressed puck/tablet.

A built-in buzzer may beep when the RFID reader has a good read; if any of the key pad's keys are depressed; the correct vaporization temperature has been met; and optionally in unison with either flashing LED—error and replace puck.

In another embodiment, a relay is configured to turn on a cooling fan. This relay could be substituted for a Triac (a triode for alternating current). A Triac is a generalized trade name for an electronic component that can conduct current in either direction when it is triggered (turned on), and is formally called a bidirectional triode thyristor or bilateral triode thyristor. Triacs are is commonly used in controlling the speed of low-power induction motors, in dimming lamps, and in controlling A.C.(Alternating Current) heating resistors. The Heater Triac turns on/off and controls the heat output of the A.C. heater.

In another embodiment, a fan provides cooling for the internal workings of the vaporizer.

In another embodiment, a heater is the heat source for the Heat Sink. An air pump delivers controllable amounts of air to the heat sink. It provides controlled amounts of vapor to the user and allows for the gassing-off of toxic carcinogens. An SCR (silicon-controlled rectifier or semiconductor-controlled rectifier) may be used to control various functions, such as turning the air pump on/off, and controlling the air output of the D.C. air pump.

### Air Flow Schematic

Shown in FIG. 1B is a schematic air flow pattern in accordance with an illustrative embodiment. Starting from the left, incoming air drawn by a pump is received through a simple foam filter. The air pump many be, for example, a high flow 24 VDC air pump is capable of delivering up to 15.0 LPM at a maximum pressure of 80 Kpa. In a preferred embodiment, it will be set to run at 9.0 LPM.

A flow meter accurately measures and controls the flow of air passed to a heating element. The flow meter may be, for example, a volumetric flow meter with temperature compensation, provides precision flow metering. The heating element may be, for example, a miniature tee type process heater capable of heating air to 220° C. Heat up time is typically less than 30 seconds.

The heated air is passed through a screen, which prevents any material from falling onto the heating element.

A tablet (puck) holder may be a magnetic, spring loaded type designed to allow for expansion, while forcing heated air through and around it for complete convection vaporization.

The vaporized air passes through another filter, before exiting the vaporizer apparatus to be inhaled by the operator. This second filter may be a user-replaceable inline air filter which filters the vaporized air before it is inhaled by the user.

In an embodiment, there is a whip or hose connected to the inline air filter for the user to inhale through. As the air is pumped out, there is no need for the whip or hose to touch the user's lips (although the user may choose to do so). This will help prevent contamination.

### Compressed Tablet (Puck)

FIGS. 2A to 2D show an illustrative compressed vaporizer tablet and optional packaging which may be used with the present vaporizer apparatus. The compressed vaporizer tablet comprises a tablet formed by compressing loose plant source material that has been processed into a compressible state. This processing may involve drying, shredding, grinding, and mixing the plant source with one or more base materials which may help the loose plant source material and any base material to bind together during compression, helping the resulting compressed tablet retain its shape.

As shown in FIG. 2C, various types of machine readable codes may be placed directly onto the compressed vaporizer tablet, whether by printing or on a label. Alternatively, as shown in FIG. 2D, the machine readable codes may be placed on packaging for the compressed vaporizer tablet. The machine readable codes may comprise optically readable codes such as bar codes or QR codes, or wirelessly detectable ID tags or codes such as RFID or other types of wireless IDs.

As an illustrative example, in an embodiment, the plant source material may be the hemp plant which is composed of approximately 20% lignin, a polymer in plants that provides rigidity. In an embodiment, as the plant source material is compressed, it is heated by frictional forces. The lignins (contained in all woody-cellulose materials) begin to flow and act as a natural glue to bind the compressed plant source materials. Sticky trichomes, which are present predominantly in flowers, will also serve to bind the material, possibly reducing the total pressure needed to form the tablets. When the compressed material exits the compression machine, the lignins cool, solidify and hold the plant source material together to form the tablet, which in a preferred embodiment is formed into a thin, perforated, cylindrical shape.

By way of illustration, one gram of plant source material can typically be compressed into approximately a 16 mm×4 mm tablet with nine 2 mm holes through the width of the tablet. The perforations or holes through the tablet should be sufficient to facilitate complete vaporization of the entire tablet when used in conjunction with the vaporizer embodiments described herein. As detailed further below, multiple tablets may be held in place in a disc cartridge for convenience and safety.

In experimentation, it has been found that hydraulic presses will generally produce compressed vaporizer tablets which are suitably dense and of sufficient hardness to retain their rigid shape. However, it will be appreciated that other types of presses (e.g. mechanical presses) may also be used if they can provide the sufficient compression force and desired ambient parameters.

By way of example, 1 gram of plant source material may be compressed into a generally cylindrical tablet of approximately 15 mm in diameter and 5 mm in height or thickness, as shown in FIGS. 2A and 2B. It will be appreciated that the amount of plant source material and the dimensions of the tablet are provided by way of illustration only, and are not meant to be limiting. For example, the compressed vaporizer tablet may be increased to 25 mm (approximately 1 inch) in diameter or even larger. Preferably, the thickness of the tablet may range from about 2 mm-6 mm, but the tablet may be thinner or thicker as may be desired.

The illustrative tablet shown in FIGS. 2A and 2B includes nine holes of approximately 1 mm in diameter. Again, this dimension is illustrative, and is not meant to be limiting. In this example, each hole is no more than about 4 mm from any other hole or an outside edge. This will mean that heat will have to penetrate no more than about 2 mm from any surface of the tablet, whether on the outside surface, or from an inner surface within one of the holes.

In an embodiment, each tablet is compressed with compression molds having at least one post or core which produces a hole in the compressed tablet. A plurality of such posts or cores may be spaced apart in the mold in order to form a pattern of a plurality of holes in the compressed tablet. The pattern of holes may be provided to align with vents provided in the vaporizer apparatus, supplying heated air into the chamber holding the compressed tablet.

The size, number, and pattern of through holes may be selected to provide varying rates of vaporization. Generally speaking, a larger number of holes will provide a greater surface area, resulting in an increased rate of vaporization of the compressed plant source material.

The compression of the plant product will prevent the usual degradation of the plant experienced from friction and bruising.

Tablets can be packaged in Child Resistant/Senior friendly blister packs or Disc Cartridges. CR/SF packaging means safety, security and convenience—preventing children from gaining access to the package's content, all while considering functionality for adults and senior citizens. Blister pack packaging is associated with true medicine.

The blister packs can be modified atmosphere packaged. Little degradation of the product will occur as each tablet will be individually sealed within the Disc Cartridge.

Each Disc Cartridge will have RFID tags attached. Product information will be encoded onto these RFID tags. This information may include product blend, THC to CBD ratio, dosing amounts, specific ailment intended alleviate and optimum vaporization temperatures.

Data can be submitted to the Minister that establishes the stability period during which after the dried plant source material is packaged, and when it is stored under its recommended storage conditions. This will allow us to include an expiry date on the packaging label.

The tablets can contain a proprietary blend of different strains of cannabis. These blends can then be tailored to alleviate specific ailments. For example a high CBD strain, as used to treat seizures, is basically rope and taste bad when vaporized. A high terpene content strain with low THC levels could be added in small amounts to improve flavor. An exact 4/1 blend of THC to CBD can be achieved with a high THC strain correctly blended with a low THC strain.

The ability to derive multiple THC to CBD ratios from only two strains will make this technology appealing to producers. No longer do they have to rely on genetics to tailor their product. The ability to tailor cannabinoid based medicines for specific diseases will allow different producers to offer standardized blends from a few base strains with differing terpene profiles.

### Illustrative Embodiments of the Apparatus

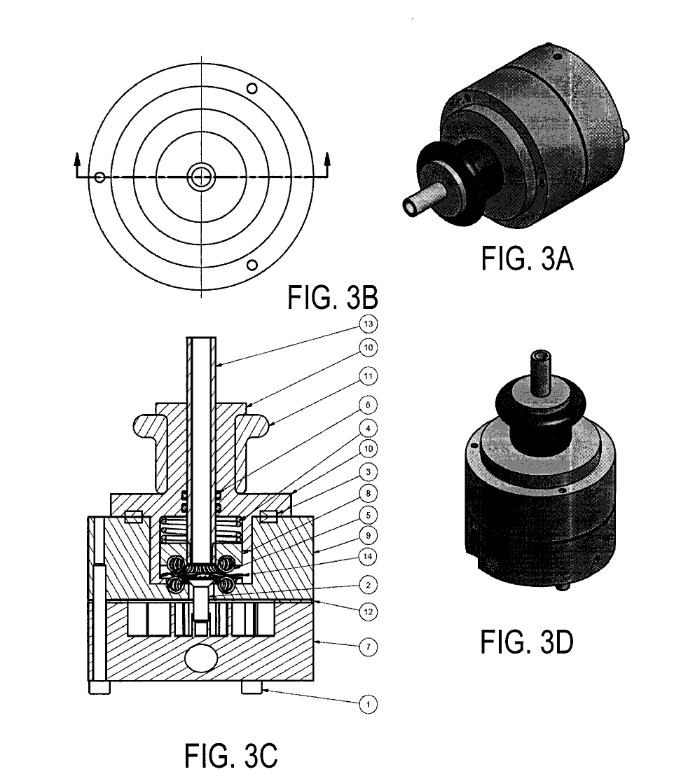

Now referring to FIGS. 3A to 3D, shown are illustrative views of an air heater assembly in accordance with an embodiment. Referring to FIG. 3C, shown is an illustrative air heater assembly with the puck/tablet holder in place.

In an embodiment, the assembly (1) includes: cap head screws (2); a countersunk head machine screw (3); magnets (4); a coil spring (5); a upper garter spring (6); two o-rings (7); a heat sink (8); an upper spring holder (9); a lower spring holder (10); an enclosure fitting (11); a rubber support (12); a heat sink gasket (13); a stem fitting and (14); the compressed puck in place.

Screws (1) hold the heatsink gasket (12) between the two halves of the heater assembly; the lower spring holder (10) and the heat sink (7) providing an air tight seal. Countersunk head machine screw (2) is placed in the center of the airflow exiting from the (7) Heatsink to channel the heated air in a circular fashion towards the tablet/puck.

Magnets (3) in the enclosure fitting (10) will align and attract to the lower spring holder (9) providing an air tight seal between pieces.

Heat sink (7) transfers heat from the internally mounted plug style electric heater to the fins. Air pumped through the Heat Sink would then be heated through convection.

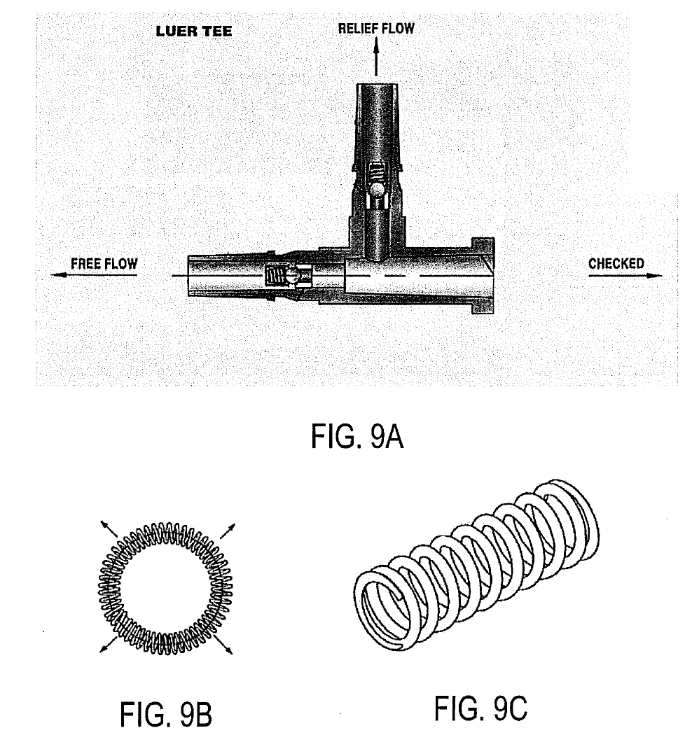

Upper spring holder (8) holds the upper garter spring. A garter spring is shown in FIG. 9B. It is between this spring and the lower garter spring the compressed perforated puck/tablet sits. This piece is attached to the stem piece (13).

Lower spring holder (9) holds the lower garter spring. It is between this spring and the upper garter spring the compressed perforated puck/tablet sits. This piece is attached to the heat sink (7) with a heat sink gasket (9) in between.

Enclosure fitting (10) has a flanged lip half way around the perimeter to allow the compressed puck to be dropped in from the unflanged side. By holding the stem fitting (13) and pulling back on the rubber support (11), the upper spring holder (8) will be retracted into the enclosure fitting (10) allowing room for the compressed puck/tablet to be dropped in. Releasing the rubber support (11) will cause the coil spring see FIG. 9C to apply sufficient pressure to the upper spring holder (8) to hold the compressed puck/tablet in place.

Rubber support (11) provides a comfortable finger grip.

Heat sink gasket (12) provides both an air tight seal and thermal insulation between the heat sink (7) and the lower spring holder (9). In an embodiment, the heat sink gasket is made of ceramic fiber insulation material which resists the flow of high temperature gas and has a maximum use temperature of about 800° F/427° C.

Stem o-rings (6) are silicone o-rings. Odorless and non-toxic, they are rated for temperatures to 450° F/232° C. These o-rings should provide addition sealing between the stem fitting (13) and the enclosure fitting (10).

Stem fitting (13) is a hollow tube which allows the heated vapor to travel from the heating cavity, created by the garter spring and the compressed puck/tablet. In this illustrative example, it terminates with a standard male luer thread.

There are two garter springs (see FIG. 9B), which fit in the curved grooves in the (8) Upper Spring Holder and (9) Lower Spring Holder. A garter spring is a coiled steel spring that is connected at each end to create a circular shape. Between these springs the compressed puck is placed. These garter spring channels the heated air in a circular fashion causing some of the air to pass over the surface of the puck numerous times.

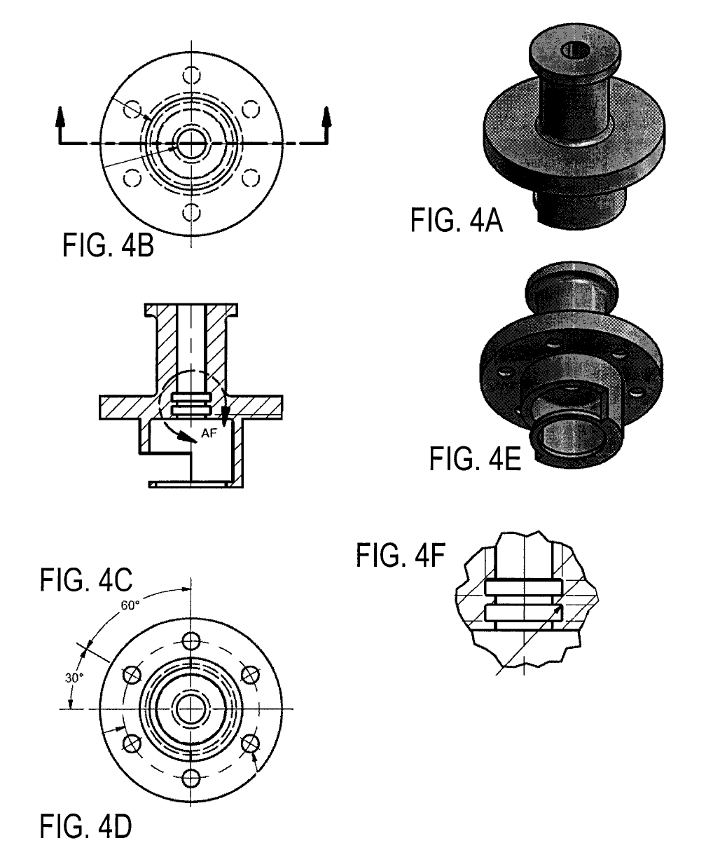

FIGS. 4A to 4F show illustrative views of the enclosure fitting (10) of FIG. 3C in accordance with an embodiment. More particularly, FIG. 4A is an overall view of the enclosure fitting.

FIG. 4B shows the various radiuses and the cut-away point shown in FIG. 4C. FIG. 4C shows the cut-away view of the enclosure. It shows how the tab that holds the compressed puck/tablet is only for 180 degrees or half the circumference. It also show the grooves for the two internal o-rings. FIG. 4D shows the placement and sizes of the holes where the six magnets will be embedded. FIG. 4E is a close up view of the internal o-rings grooves.

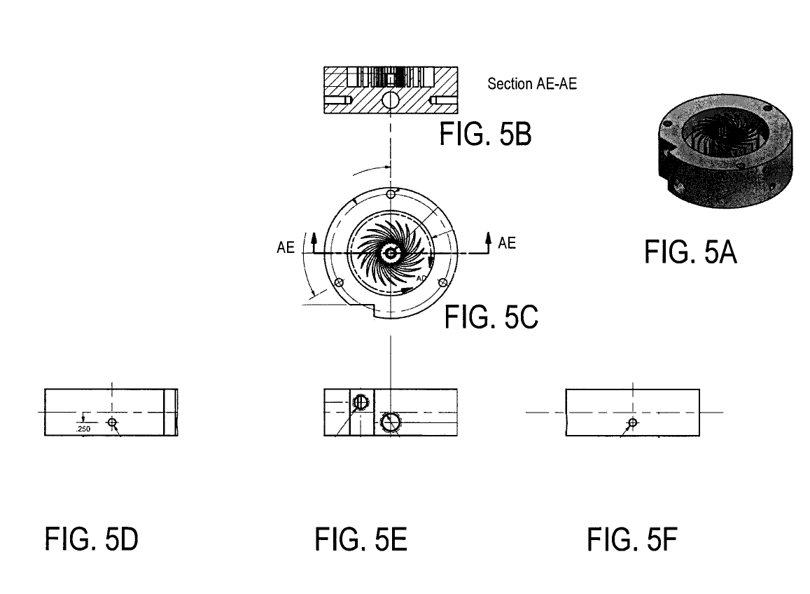

FIGS. 5A to 5G show illustrative views of a heat sink in accordance with an embodiment. As best shown in FIGS. 5A and 5C, a circular arrangement of fins provide a greater surface area to provide a heat sink absorbed by the component. Using thermal conduction the heater transfer heat to the entire heat sink. The increased surface area of the fins provides an increased heat transfer through conduction and convection. The fins also provide turbulence to assist conduction heat transfer.

FIGS. 5E and 5G show the threaded hole where the temperature sensor and high limit thermostat are mounted to the side of the heat sink. FIG. 5F shows two threaded holes. The top left one, which is against the flat notched surface, is where the barb fitting for the air in will be mounted. The notch is provided to allow access for a wrench used when tighten the barb fitting. The center bottom one is where the plug style heater is inserted and threaded into place.

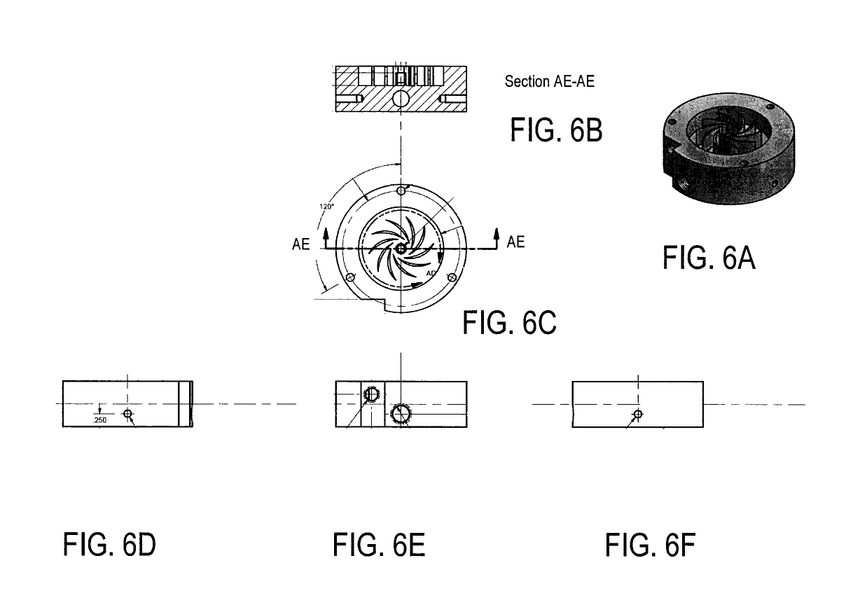

FIGS. 6A to 6G show illustrative views of an alternative heat sink, in which the pattern of fins is rearranged with fewer fins.

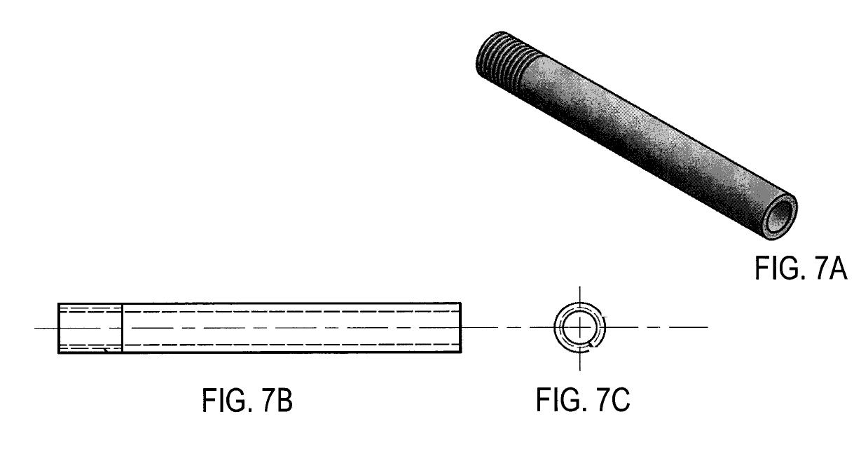

FIGS. 7A to 7C show illustrative views of a hose connection pipe in accordance with an embodiment. In this illustrative embodiment, the hose connection pipe includes a standard luer fitting, which allows a luer tee fitting to be tightened onto it. This type of luer taper is a standardized system of small-scale fluid fittings used for making leak-free connections between a male-taper fitting and its mating female part on medical and laboratory instruments.

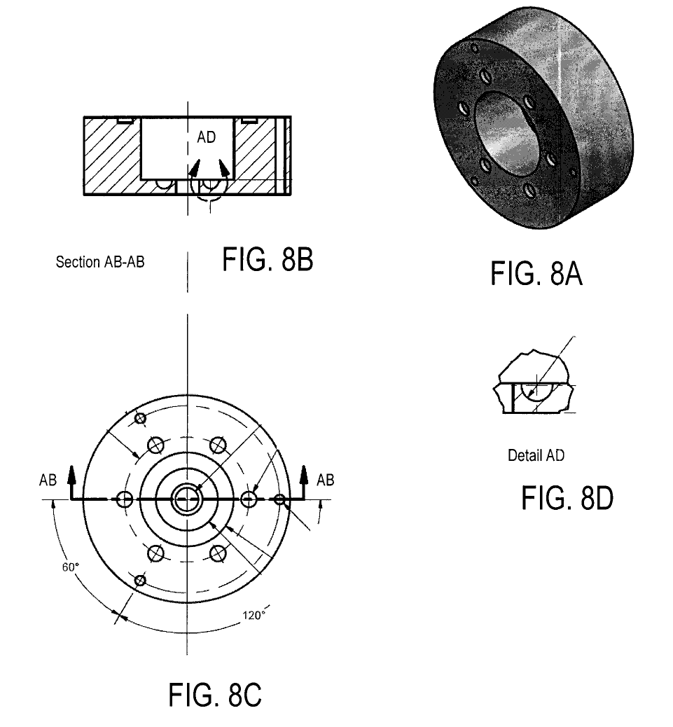

FIGS. 8A to 8D show illustrative views of a lower spring holder in accordance with an embodiment. More particularly, FIG. 8B shows the circular channel for the garter spring FIG. 9B. The cavity is where the (8) Upper Spring Holder should travel. FIG. 8C shows the placement and sizes of the holes where the six magnets will be embedded. It also shows the three threaded mounting holes. It also shows the hole where the heated air enters in the center. FIG. 8D details how the grove is slightly more than 180 degrees. This is so once the garter spring is popped into the groove it does not easily come out. It is between the coils of this spring and its mate in the (9) lower spring holder, where the compressed puck/tablet is held. Springs are used to minimize heating of the compressed puck/tablet through conduction.

In an embodiment, the vaporizer apparatus utilizes a spring loaded compressed tablet access chamber. Removing the compressed tablet (puck) holder, comprised of the enclosure fitting with the stem piece, coil spring, upper spring holder, rubber support and garter spring installed, from the heating unit comprised of the heat sink, heat sink gasket and lower spring holder, and garter spring installed, and holding it on its side with the unflanged portion of the enclosure fitting pointing down, while depressing the rubber support will allow the tablet to fall out. There is no need for the user to touch a spent tablet.

In an embodiment, a user replaceable air filter is placed after the tablet holder to prevent any solid materials from being inhaled. Since the vaporizer apparatus monitors air flow and the current draw of the air pump, it will notify the user when to clean the unit and replace the air filter.

In an embodiment, an onboard microprocessor keeps a record each use with time, temperature settings, and dose amounts. When the memory is full or after a defined time period, the vaporizer apparatus may automatically connect to the network database through GSM or may instruct the user to connect the device to the internet through its LAN port. Once connected to a network database server, usage information may be uploaded to the database. This database may then collect usage information, and allow medical researchers to monitor effectiveness and validity of dosing amounts.

### Sensors

In an embodiment, the vaporizer apparatus may include a number different types of sensors. For example, temperature sensors monitor the air exiting the heating chamber. Two sensors may be utilized for redundancy.

In another embodiment, an axis accelerometer may be used to detect if the unit is accidentally tipped over or dropped, automatically turning off the unit. This will also prevent the unit being used in anything but a proper upright position.

In another embodiment, a magnetic or light sensor may be configured to detect when the compressed tablet (puck) holding chamber has been removed. The machine will not operate and the heater will not heat while this tablet holding chamber is removed. This sensor will also tell the unit when the puck has been replaced for tracking purposes.

In another embodiment, a second magnetic or light sensor will detect when the lid to the machine is open or closed, allowing for auto-on when the lid is open and auto-off when the lid is closed.

In another embodiment, sensors provided in the compressed tablet (puck) holding chamber allow the vaporizer unit to detect the type of compressed tablet that has been received.

In another embodiment, sensors detect machine readable label, either provided on the compressed tablet or on packaging for the compressed tablet in order to detect the type of compressed tablet.

In another embodiment, a volumetric flow meter will provide precision flow metering with temperature compensation. This meter will allow for precise consistent dosing of the vaporized product.

In another embodiment, the vaporizer apparatus includes an LCD display which may indicate the various functions and states of the apparatus. These may include, but are not limited to: Power on Memory full, connect to LAN; Heating Temperature (set); Cooling Missing puck holder; Low battery Unit plugged; Clean machine; Replace puck Dispensing dose; Replace filter; Powering off; Download memory, connect to LAN; Press enter.

In an embodiment, various user interface controls may be provided on the vaporizer unit. To simplify operation, the controls may be limited to a few navigating buttons, such as Up, Down, Left, Right, and enter. These controls may be provided as manual buttons, or alternatively as touch screen buttons on a touch screen display.

The vaporizer unit may be powered by an onboard battery, or alternatively connected to an AC outlet by a power cord and transformer.

In another embodiment, the compressed vaporizer tablet may include a blend of different plant source materials selected to alleviate specific conditions or ailments. These blends may be selected based on the active ingredients found in each plant source material, and the amount of each plant source material in the blend is proportional to the desired proportion of active ingredients.

As an illustrative example, THC (tetrahydrocannabinol) and CBD (cannabidiol) are the two most prominent chemical compounds in the cannabis plant, and are often found together in certain ratios. Illustrative examples of THC to CBD ratios include the following:
88:1 Non-psychoactive. Charlotte's Web is a well know example of a CBD dominant strain. Used to treat children with severe epilepsy and Dravet syndrome.
18:1—Non-psychoactive. Some patients find CBD dominant medicines helpful for anxiety, depression, psychosis and other mood disorders.
8:1—Non-psychoactive. Some patients find mid-range CBD:THC ratios helpful for spasms, convulsions, tremors, endocrine disorders, metabolic syndrome and overall wellness.
4:1—Borderline psychoactive. For patients who have some tolerance for THC. Some patients find mid-range ratios helpful for pain relief, immune support and other health benefits. Has been found to kill all forms of cancer cells in a Petri dish.
2:1—Psychoactive in larger doses. For patients who have some tolerance for THC. Some patients find balanced ratios helpful for inflammation, chronic pain, gastrointestinal issues and stress relief.
1:1—Psychoactive. For patients who tolerate THC well. Some patients find a balanced ratio helpful for neuropathic pain, rheumatism and overall mood enhancement.

A patient's sensitivity to THC (tetrahydrocannabinol) is a key factor to determining the appropriate ratio and dosage of high CBD cannabis medicine. CBD can lessen or neutralize the intoxicating effects of THC. So a greater ratio of CBD-to-THC means less of a “high.” But CBD-dominant cannabis remedies with little THC, while not intoxicating, are not necessarily the most effective therapeutic option. That's because CBD and THC heighten one another's medicinal effects. A combination of CBD and THC will likely have a greater anti-cancer effect or analgesic (painkilling) effect, for example, than CBD or THC alone.

Depending on the THC to CBD ratio, the microcontroller may be programmed to utilize a particular metered air flow, heating profile, and delivery time in order to control the dosage of therapeutic compounds from the vaporized, compressed tablet.

For example, THC evaporates at 157° C., while CBD evaporates between about 160° C. to 180° C. Therefore, by controlling the temperature to be below 160° C. but above 157° C., the vaporizer unit can promote or inhibit vaporization of various constituent compounds.

Other examples of cannabinoid evaporation temperatures include:

Delta-8-THC—175-178° C.

CBN—185° C.

CBC—220° C.

THCV—220° C.

Flavinoids found in cannabis plants include the following, with their evaporation points:

Beta-sitosterol—134° C.

Apigenin—178° C.

Cannflavin A—182° C.

Quercetin—250° C.

Terpenoids found in cannabis plants include:

Beta-caryophyllene—199 C.

Alpha-terpinol—156° C.

Beta-rnyrcene—166-168° C.

Delta-3-carene—168° C.

1,8-cineole—176° C.

D-limonene—177° C.

P-cymene—177° C.

Linalool—198° C.

Terpinol-4-oi—209° C.

Borneol—210° C.

Alpha-terpineol—217° C.

Pulegone—224° C.

Depending on the evaporating temperature of the compound desired to be promoted or inhibited, the microprocessor of the present vaporizing apparatus can be set to provide a metered air flow and temperature profile which best achieves the desired vaporization or inhibition.

By way of example, as it is known that the boiling point of benzene is 80.1° C., and 110° C. for toluene, the vaporizer may be temporarily be set to a “gassing-off” mode to inhibit benzene and toluene in the vapor prior to being inhaled. As illustrated by way of example in FIG. 9A, while in a gassing-off mode, an air pump is configured to engage at low pressure at those temperatures so the air/vapor flow would be directed to the atmosphere (relief flow) and not down the whip (free flow) for inhalation. The relief valve may incorporate properly recalibrated springs to allow this gassing-off to occur.

The vaporizer of FIG. 9A may be used for either compressed tablets or loose fill plant source material contained in a mesh container as described in further detail below. FIG. 9B shows a detailed view of a coil spring (see FIG. 3C) and FIG. 9C shows a detailed view of a compression spring, respectively.

In an embodiment, the relief valve is normally open and closes at pressures greater than 3 psi, and normally closed and opens at pressures greater than 3 psi. Pumped vapor then goes into a checked end. Low pressure vapor, produced when the unit is gassing-off, is vented to atmosphere through a relief flow end. When the gassing-off is finished, a high pressure vapor is directed out the free flow end to the user for inhalation. Advantageously, this relief valve significantly reduces the risk of inhaling benzene and toluene, and other contaminants.

### Loose Fill Embodiment

Most existing vaporizers require the plant source material to be loosely packed. However, with these prior art devices, measuring a consistent dosage of loose fill plant source material may be challenging, as the amount of material that may be placed into a volume of space may depend on various factors including the size of the loose fill materials and the amount that the plant source material is packed. This creates significant inconsistencies between dosages if attempted manually.

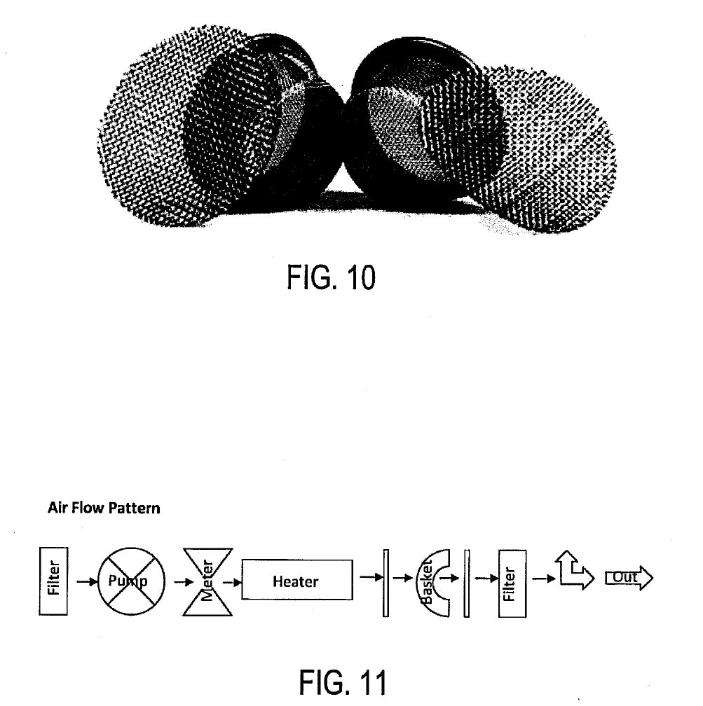

As shown by way of example in FIG. 10, different sizes of mesh containers or baskets and screens may be used to filter different sizes of loose plant source materials. In order to measure a consistent amount of loose fill plant source material, a mesh basket and screen may be used to obtain relative consistency in the loose fill plant material size and volume of material in the mesh basket.

In an embodiment, instead of stainless steel, the mesh container or basket and screen disc may be constructed of hemp fibre, or other nontoxic, nonflammable materials that may contain the loose fill plant source material for a single use. The single use molded mesh baskets hold the loose fill plant source material, and allow the loose fill plant source material to be packaged individually, for example in blister packs. In an embodiment, the molded mesh basket may be adapted to disintegrate, such that it can be removed from the vaporizer chamber together with any residue remaining after vaporization.

Shown in FIG. 11 is an illustrative air flow in accordance with the present loose fill embodiment. Starting from the left, HEPA filtered air is fed through a precision flow meter before entering a gas or electric heating chamber. A battery or a gas/fuel may be used as a heat source in a portable version. As an illustrative example, the gas used may be Butane, Naphtha, or charcoal lighter fluid. Electricity may be used to generate heat for a table top version. The parts shown in this illustrative embodiment include the following:

HEPA Filter Air Pump Flow meter Heater Screens Loose Fill Basket filter off gassing out.

HEPA Filter—Incoming air is first filtered through this HEPA filter.

Air Pump—A high flow 24VDC air pump is capable of delivering up to 15.0 LPM at a maximum pressure of 80 Kpa. It will be set to run at 9.0 LPM.

Flow Meter—Volumetric flow meter with temperature compensation, provides precision flow metering, measuring the amount of air passing through the heater and loose fill plant source material to the user. Thus, equal metered doses can then be consistently administered to the user.

Heater—The heater is capable of increasing air temperature to 220 degrees C. Heat up time is typically less than 30 seconds. Precision temperature sensors monitor the air exiting the heating chamber. Air speed and element temperature, controlled through the microprocessor, allows for temperature stability to +/−3 degrees C. As the unit monitors the total amount of heated air going through the loose fill chamber, and knows when loose fill plant source material is placed in the chamber (through addition sensors) the unit can notify the user as to when to replace the loose fill.

Screen—These screens prevents any plant source material from falling into or out of the heating chamber.

Loose fill basket—The extremely fine stainless steel mesh screen bowl holds the loose fill plant source material.

Filter—A disposable inline air filter filters the air before it is inhaled by the user.

Off Gassing—This is where the toxic carcinogens Benzene and Toluene are first vented to the atmosphere. Although vaporization eliminates the formation of most carcinogens related to combustion, because of their low boiling points, small amounts of benzene and toluene—known carcinogens—would still be present in the vapor. Their boiling points are 80.1 C for Benzene and 110 C for toluene. The range of temperature in which all cannabinoids, terpenes, and flavinoids evaporate lies between 134 and 220 degrees Celsius.

In an embodiment, the Vaporizer heats the intended loose fill plant source material to just over 110C (110-120C) to remove potential carcinogens, making the vapor safer to inhale.

The air pump engages at low pressure at those temperatures so the air/vapor flow would be directed to the atmosphere and not down the whip for inhalation.

Out—In an embodiment, an optional whip or hose may be connected to the inline air filter for the user to inhale through. As the air is pumped out, there is no need for it to touch the user's lips (although the user may choose to do so). This will help prevent contamination.

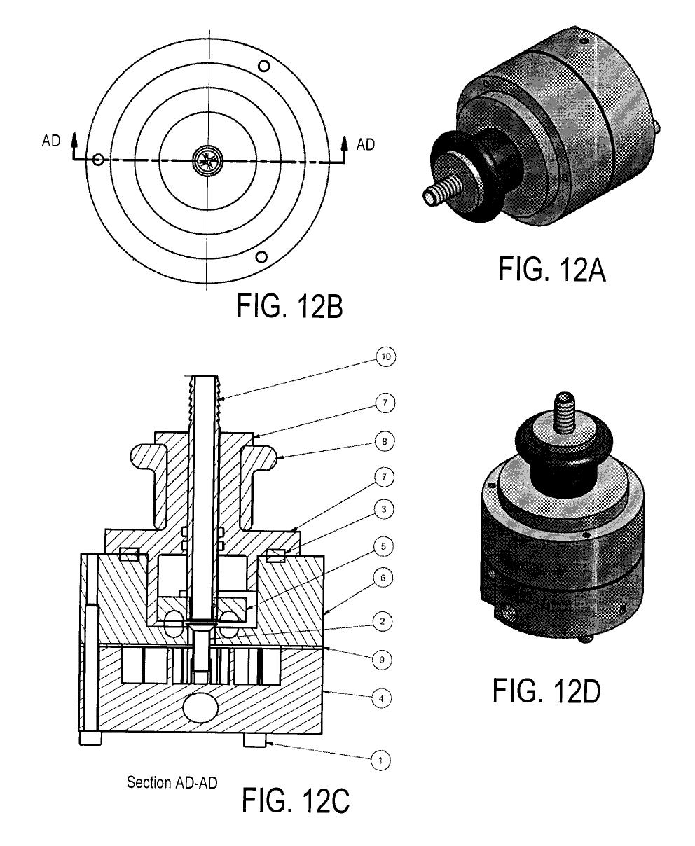

Now referring to FIGS. 12A-12D, in an embodiment, the loose fill vaporizer utilizes a spring loaded loose fill access chamber. This illustrative example includes the following parts:
1) Screws—hold the (9) Heatsink Gasket between the two halves of the heater assembly; the (6) Lower Spring Holder and the (4) Heat Sink providing an air tight seal.
2) Countersunk head machine screw—This screw is placed in the center of the airflow exiting from the (4) Heatsink to channel the heated air in a circular fashion towards the tablet/puck.
3) Magnets—magnets in the (7) Enclosure Fitting will align and attract to the (6) Lower Spring Holder providing an air tight seal between pieces.
4) Heat Sink—transfers heat from the internally mounted plug style electric heater to the fins. Air pumped through the Heat Sink would then be heated through convection.
5) Upper Spring holder—Provides pressure against the formed screen. This piece is attached to the (11) Stem Piece.
6) Lower Spring Holder—Hot air exits the heat sink through this piece. This piece is attached to the (4) Heatsink with a (9) Heatsink Gasket in between.
7) Enclosure Fitting—has a flanged lip half way around the perimeter to allow the loose fill basket and screen to be dropped in from the unfledged side. By holding the (11) Stem Fitting and pulling back on the (8) Rubber Support, the (5) Upper Spring holder will be retracted into the Enclosure Fitting allowing room for the loose fill basket and screen to be dropped in. Releasing the Rubber support will cause the coil spring to apply sufficient pressure to the Upper Spring Holder to hold the loose fill basket and screen in place.
8) Rubber Support—provides a comfortable finger grip.
9) Heatsink Gasket—provides both an air tight seal and thermal insulation between the (4) Heatsink and the (6) Lower Spring Holder. Made of ceramic fibre insulation material it resists the flow high temperature gas and has a Maximum Use temperature of 800F/427C.
10) Stem Fitting—This hollow tube allows the heated vapor to travel from the heating cavity. It terminates with a standard male LUER thread.

Removing the loose fill holder from the base unit and holding it on its side while depressing the holder will allow the loose fill to fall out. Thus, there is no need for the user to ever touch spent loose fill plant source material.

Advantageously, the chamber illustrated in FIGS. 12A-12D is capable of receiving a compressed tablet, such that either a compressed tablet or loose fill plant source material contained in a mesh basket may be used for the same vaporizer. In an embodiment, for compressed tablets, a garter spring may be used in both upper and lower spring holders. For mesh baskets, such garter springs need not be used. This may provide users with more options for receiving a dosage, depending on the condition being treated and the preference of the user.

In an embodiment, a user replaceable air filter may be place after the loose fill holder to prevent any solid materials from being inhaled. As the vaporizer unit monitors air flow and the current draw of the air pump, the vaporizer can notify the user when to clean the unit and replace the air filter.

As noted earlier, an onboard microprocessor may keep a record each use with time, temperature settings, and dosage amounts. When the memory is full, or after a defined time period, the device may be connected to the internet through a LAN port, or to a computer via a USB port, for example. Once connected the device can communicate with a website and all the stored information can be uploaded to a database at the website. This website database can then become a powerful tool for assisting doctors with the efficacy and the validity of dosage amounts for treading various conditions.

In an embodiment, temperature sensors will monitor the air exiting the loose fill heating chamber. Two sensor are utilized for redundancy. These sensors are ultrafast acting.

A three-axis accelerometer may be used to detect if the unit is accidentally tipped over or dropped automatically turning off the unit. It will also prevent the unit being used in anything but the proper upright position.

In an embodiment, a magnetic sensor may also be used to detect when a loose fill basket has been removed from the chamber. As a safety feature, the vaporizer will not operate and the heater will not heat while this piece is not found in the chamber. This sensor will also tell the unit when the loose fill has been replaced for tracking purposes.

In an embodiment, a second magnetic sensor detects when the lid to the vaporizer is open or closed, allowing for auto-on when the lid is open and auto-off when the lid is closed.

In an embodiment, loose fill plant source material can be packaged in Child Resistant/Senior friendly blister packs. CR/SF packaging means safety, security and convenience—preventing children from gaining access to the package's content, all while considering functionality for adults and senior citizens. Blister pack packaging is associated with true medicine.

The blister packs can be modified atmosphere packaged. Little degradation of the product will occur as each one gram puck is individually packaged.

Data can then be submitted to regulatory authorities that establishes the stability period during which after the dried plant source material is packaged, and when it is stored under its recommended storage conditions. This will allow us to include an expiry date on the packaging label.

The loose fill will contain a proprietary blend of different strains of cannabis. These blends can then be tailored to alleviate specific ailments. For example a high CBD strain, as used to treat seizures, is basically rope and taste bad when smoked. A high terpene content stain with low THC levels could be added in small amounts to improve flavor.

### Table Top Embodiment

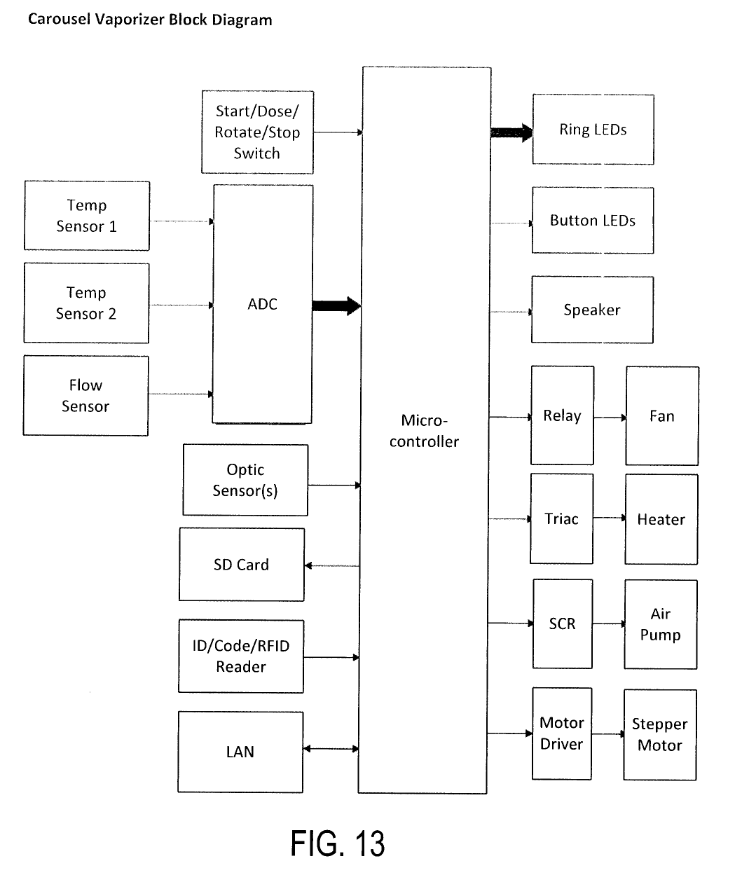

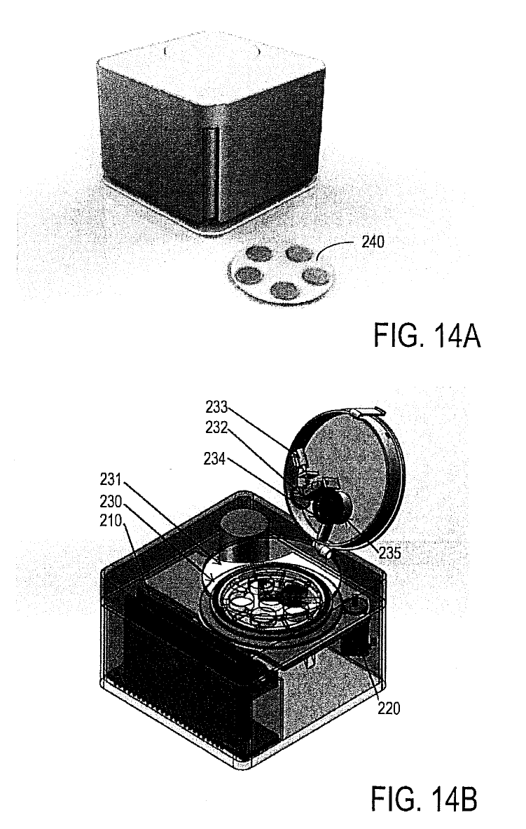

Now referring to FIG. 13, shown is a schematic block diagram of a vaporizer in accordance with another illustrative embodiment, in this case a table top model. In this table top embodiment, the vaporizer of FIG. 13 may be embodied in a vaporizer device as illustrated in FIGS. 14A and 14B, which includes a carousel adapted to receive a disc cartridge of compressed vaporizer tablets.

As shown in FIG. 14B, in a partial see-through view, various components may include a carousel holder holding a carousel, which is adapted to receive a disc cartridge. A carousel drive motor is adapted to rotate the carousel holder to position a compressed vaporizer tablet, as described in further detail below. A hinged lid may include a mechanism for forming perforations in a disc cartridge, including a top perforator lock solenoid, and a top perforating cone rotatable by a gear. The hinged lid may also include a vapor receiving vessel, which is sealed to the top of a compressed vaporized tablet when it is rotated into position in the carousel.

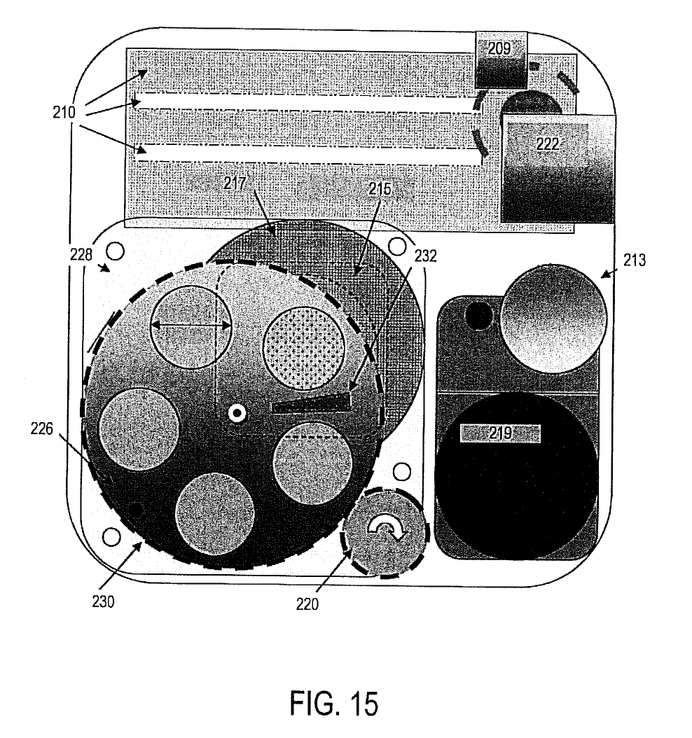

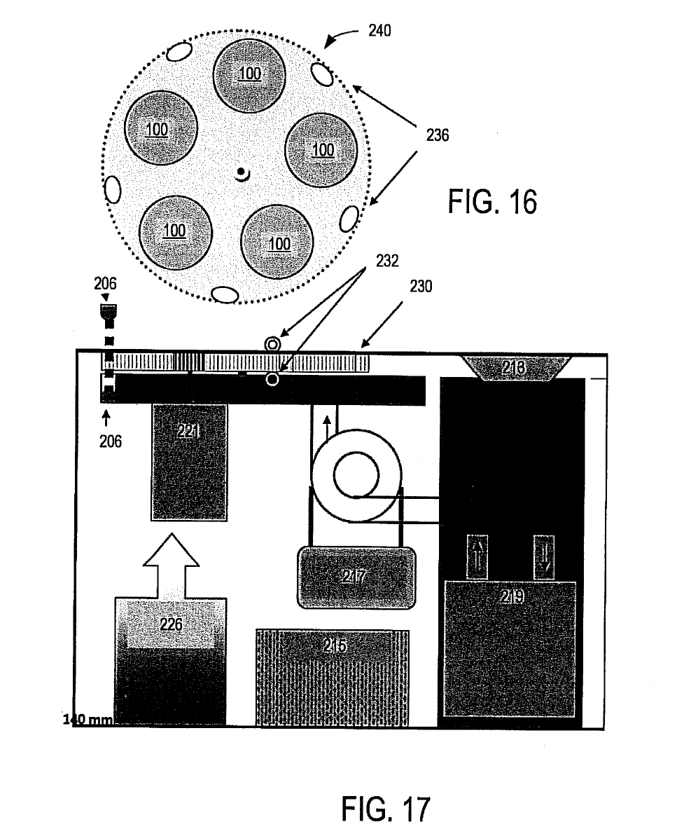

FIG. 15 shows a schematic top view of various components within the table top vaporizer of FIGS. 14A and 14B, and FIG. 16 shows a front view of an illustrative disc cartridge of compressed vaporizer tablets in accordance with an embodiment.

FIG. 17 shows a schematic side view of various components within the table top vaporizer of FIGS. 14A and 14B.

In this embodiment, compressed tablets are held in place in a disc cartridge. As each tablet is spent, the device rotates the disc cartridge to the next available unused tablet. When all the tablets in the disc cartridge are spent the device notifies the user to replace the entire disc cartridge.

In an embodiment, the device does not rely on a fan or inhalation to move the heated air through the tablets but instead incorporates an air pump. HEPA filtered air from this air pump is fed through a precision flow meter before entering the electric heating chamber. The flow meter indirectly controls the volume of airflow and duration of the air pump. Precise metered doses can then be consistently administered to the user.

In an embodiment, one or more precision temperature sensors monitor the air temperature entering and exiting the CPT vaporization chamber. Microprocessor controlled temperatures, air speed and air volume should allow air vaporization temperature stability of +/−3° C. and a minimum temperature drop across the tablet of 3° C.

The MDI not only monitors air flow but also the air pump's current draw, making it possible to notify the user of possible blockages or to replace the air filter.

In an embodiment, an RFID reader installed in the MDI will read prescription and other relevant information from the Disc Cartridge to set the vaporization dosing parameter. In case there is no RFID information the vaporizer will default to the factory settings. All information will be stored in the imbedded PC.

The imbedded microprocessor will keep a record each use with time, temperature settings, and dose amounts and other data from the RFID tag. When the memory is full or after a defined time period, the unit will inform the user to connect the device to the internet through its LAN port or to remove the memory card and place it into the user's computer card reader.

In an embodiment, a secure website server may be set up to collect vaporizer usage data in a database. Device access to the website may be secured, for example, through recognition by the server of a valid vaporizer device. Once connected, anonymized usage data may be uploaded to the website. This database will become a powerful tool in assisting doctors in the effectiveness of validity of dosing amounts. It will allow the design of next generation effective cannabinoid medicines based on these results.

In an embodiment, a cooling fan may be used to cool the device as needed to maintain a suitable operating temperature.

Since the disc holder needs to be affixed to the heater to provide a good seal and good fit, and the drive motor affixed to provide a good fit to the carousel, a mounting plate will be used to attach those and other items. The mounting plate will be of a material that does not transfer heat while remaining rigid, such as Ceramic, Bakelite, reinforced Teflon etc. This will allow the other parts to be mounted to it without significant heat transfer.

In an embodiment, the device includes a carousel for receiving a disc cartridge, in which a number of compressed tablets are packaged. A suitable stepper motor controlled by the microprocessor may be used to rotate the carousel. The edge of the carousel is toothed as is the drive motor. In an illustrative embodiment, the disc pack has dimensions of approximately 2¾″ (7cm) in diameter, and approximately ⅜″(5 mm) in height. As shown in this illustration, there are five positioning notches protruding in from the edge of the carousel that corresponds to a notch in the disc cartridge ensuring proper alignment. There is a hole in each of the carousel's five positioning notches, and each hole is read by the optic sensor to ensure the product tablet is properly aligned with the heater exit hole after each rotation.

An IR emitter will be placed in the lid to project a beam through the carousel to the IR detector mounted underneath. This will align the cavities in the carousel with the heater output.

Two perforating wheels impart perforations upon the “ next-to-be-used” cavity in the strip pack. A lower perforating wheel will be mounted to the mounting plate to provide perforations to the underside of the strip pack. An upper perforating wheel will impart perforations to the top of the strip pack. It will be located in the Lid/Lever. It will be spring loaded as well to compensate for height variances.

In an embodiment, one or more circuit boards may be used to control the device. For example, a connector power board may include a power in connector, LAN connector, board connectors for other boards, a power supply and possibly the power relays. The same circuit board or another board may include a main CPU and control the drive motor, heater, air pump and LED lights. The same circuit board or another board may also input the switches, temp sensors and the position sensor. Additional features on the same circuit board or another board may include a machine reader, such as an ID/Code/RFID Reader, a memory card for storage, and user dedicated memory.

In an embodiment, a speaker or buzzer could be mounted under the top plate above the air pump or circuit boards or attached to the main board.

In an embodiment, an inline HEPA filter may be used to filter out noxious compounds in the vapor. For example, a PureFlo® (MJ) Junior Cartridge manufactured by ZenPure Americas Inc. of Manassas, Va., USA may be used. Possible access to this filter could be obtained through the bottom of the desk top device, next to the cooling fan.

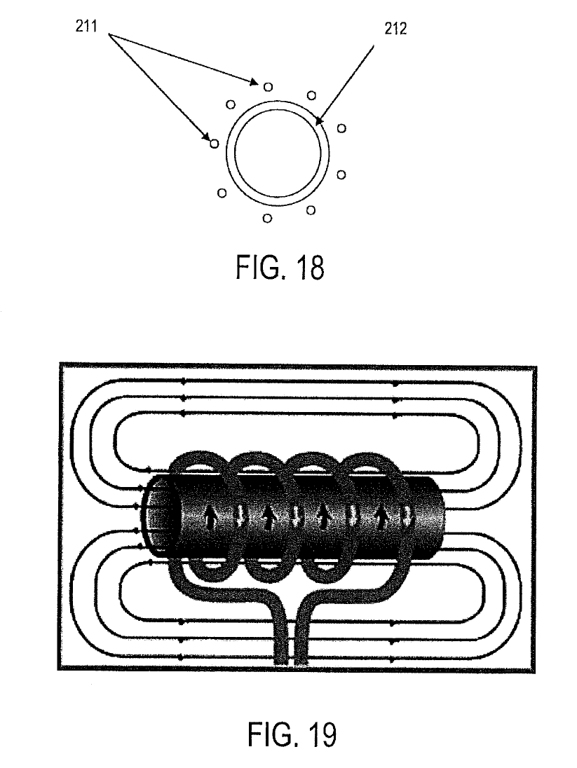

FIG. 18 shows a schematic diagram of LED indicators which may be provided on the table top vaporizer of FIGS. 14A and 14B.

| FUNTION | RING LED'S (SINGLE COLOR) | BUTTON LED'S (TRI-COLOR) |
|---|---|---|
| OFF | OFF | SOLID RED |
| HEATING | ON SEQUENTIALLY AS | PULSING GREEN |
| | TEMPERATURE RISES | |
| DEGASSING | STOP AT DEGASSING TEMP (3 LED'S) | PULSING GREEN |
| READY | OFF | SOLID GREEN |
| DOSING | ON SEQUENTIALLY AS DOSE IS | SOLID GREEN |
| | DELIVERED | |
| REPLACE | EVERY 3RD LED SEQUENTIALLY | PULSING RED |
| TABLET | FLASHING | |
| MEMORY | OFF | PULSING BLUE |
| FULL | | |
| CONNECTED | CHASING | SOLID BLUE |
| TO | | |
| INTERNET | | |
| READING | OFF | SINGLE BLUE FLASH |
| RFID DATA | | |

Beep: The speaker may be set to beep upon the occurrence of various events, such as:
   On (button press) “power on”.
   On (button press) “dosing”.
   On replace tablet error and every 30 seconds until replaced.
   On good RFID read.
   On memory is full error, every ten minutes while power is on, or once an hour when power is off until connected to internet.
   On good connection to internet.
   On (button press and hold) “power off”.
Button press sequence:
   Press, while lever closed, to turn on and start heating.
   Press, while lever closed, to start dosing—can be repeated.
   Press, while lever open, to rotate tablet holder (multi tablet loading).
   Press and hold, while lever closed, to shut off. Auto shut off after ten minutes or so.

FIG. 19 shows a schematic diagram of a magnetic induction heater in accordance with an embodiment. Induction heating is a fast, efficient, precise, repeatable, non-contact method for heating metals or other electrically-conductive materials, and offers an attractive combination of speed, consistency and control. The efficiency of an induction heating system for a specific application depends on several factors: the characteristics of the part (to be heated) itself, the design of the inductor, the capacity of the power supply, and the amount of temperature change required for the application. The size of the induction power supply required for heating a particular part can be calculated based on how much energy needs to be transferred to the work-piece. This depends on the mass of the material being heated, the specific heat of the material, and the rise in temperature required. Heat losses from conduction, convection and radiation should also be considered. Finally, the efficiency of induction heating for specific application depends on the amount of temperature change required. A wide range of temperature changes can be accommodated; as a rule of thumb, more induction heating power is generally utilized to increase the degree of temperature change. It is within the inductor that the varying magnetic field required for induction heating is developed, through the flow of alternating current. Temperature uniformity within your part is achieved through correct inductor design. The most effective uniformity can be achieved in round parts. There is a proportional relationship between the amount of current flow and distance between the inductor and part. Placing the part close to the inductor increases the flow of current and the amount of heat induced in the part. This relationship is referred to as the coupling efficiency of the inductor.

As an illustrative example, the inductor may comprise 5 turns of 6 mm O.D. borosilicate glass tubing coated with Aremco 597-C High Temp Silver filled Coating. Borosilicate glass is a type of glass with silica and boron trioxide as the main glass-forming constituents. Borosilicate glasses are known for having very low coefficients of thermal expansion (˜3×10−6/° C. at 20° C.), making them resistant to thermal shock, more so than any other common glass. Aremco 597-C has a Thermal Conductivity of 9.1 W/m-K and a 1700 F (927 C) Continuous Service Temperature. At 0.0002 ohm-cm Volume Resistivity, it should allow for good heat transfer and electrical conductivity. Aremco 597-A could be used to bond the inductor tube to the lead wires.

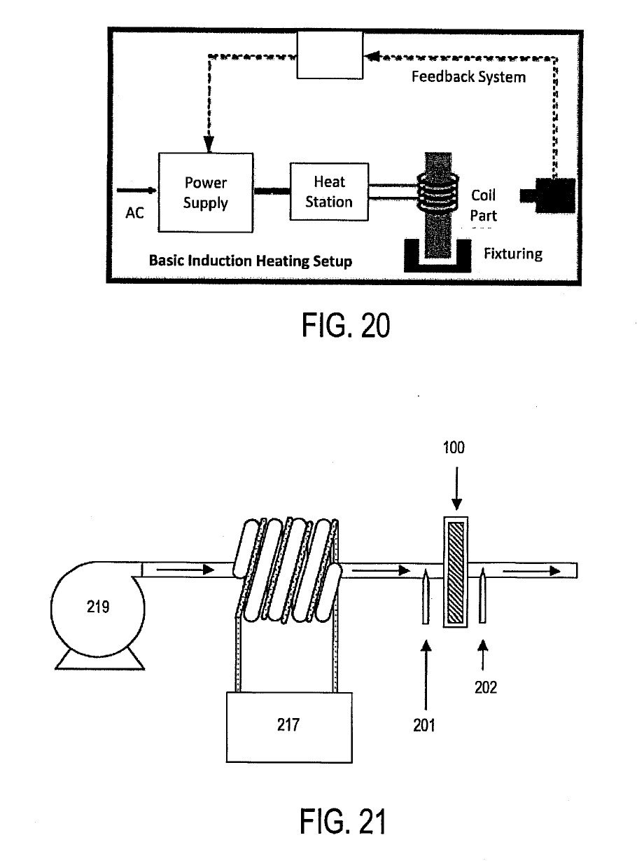

FIG. 20 shows a schematic block diagram of a basic induction heating subsystem in accordance with an embodiment. Inductors are often made of copper tubing—a very good conductor of heat and electricity—with a diameter of ⅛″ to 3/16″; larger copper coil assemblies are made for applications such as strip metal heating and pipe heating. Inductors are usually cooled by circulating water, and are most often custom-made to fit the shape and size of the part to be heated. So inductors can have single or multiple turns; have a helical, round or square shape; or be designed as internal (part inside inductor) or external (part adjacent to inductor). FIG. 21 shows a schematic block diagram of the induction heating subsystem of FIG. 20 heating a flow of air from an air pump to heat a compressed vaporizer tablet in accordance with an embodiment.

The heater core preferably has enough mass to allow for extremely fast heating and still provide rapid cooling. A borosilicate glass helical tube inductor will used as the heat exchanger. The number of coils and dimensions will need to be verified. Preferably, the heat exchanger should reach operational temperature within forty five seconds, and provide stable air temperatures exiting the helical tubing in both low flow and high flow applications. The heater should cause heated air in the helical tube to reach over-temperature between uses. If this is a problem then (periodic) continuous air flow through the glass helical coil may be an option. Induction coil and driver circuitry should be of minimal physical size as room is limited. Induction coil should be matched and use the best available parts to allow for maximum power output for size. The helical heat exchanger tube should attach directly to the holder of the Perforated Tablet.

Kovar, Dilver P, or Fernico 1, are FeNiCo alloys that have the same expansion behaviour as borosilicate glass, and because of that are used for optical parts in a wide range of temperatures and applications, such as satellites. It is available in sheet, rod, insulated wire, foil, tubing, powder, and fabricated shapes. A tube 30 mm long, 5m -10 mm O.D. with 1 mm wall would be a starting place for the size of the core.

The CPT holder needs to be bonded directly to the borosilicate glass helical inductor. It will also hold the temperature sensor(s). For this reason it should have the same thermal expansion as the glass to prevent breakage at the join. Temperature sensors will need to be installed both before and after the CPT Holder for best Induction Heating Control.

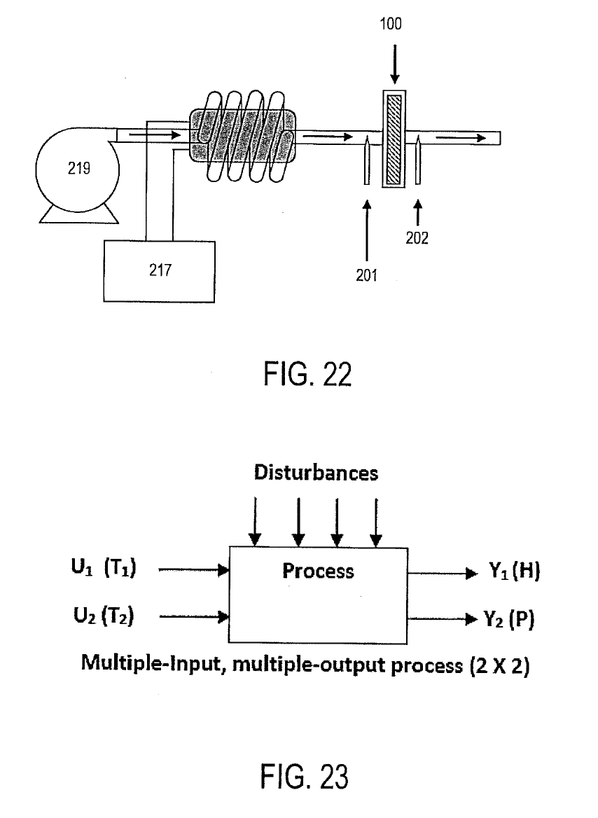

FIG. 22 shows a schematic block diagram of an alternative heating subsystem which utilizes a plug style heater rather than an induction heating design. As will be appreciated, various different types of heating subsystems may be used instead of an induction heating subsystem as described above.

FIG. 23 shows a schematic block diagram of a control subsystem in accordance with an embodiment for controlling multivariable processes in the system. For example, it may be desirable for the temperature drop between T1 and T2, to be within an adjustable window. Based on the vaporization temperatures (shown below), the range of achievable temperatures should be from; 157-200° C. min. to 135-220° C. max. The settable window range should be 2-20° C. Increasing the airflow through the helical coil heat exchanger increases temperature of the airflow. Temperatures based on air flow after an initial heat up time of 37 to 50 seconds (See test 3) ranged 140-220° C. (Fixed on/off pulse rate of 0.2/0.5 seconds).

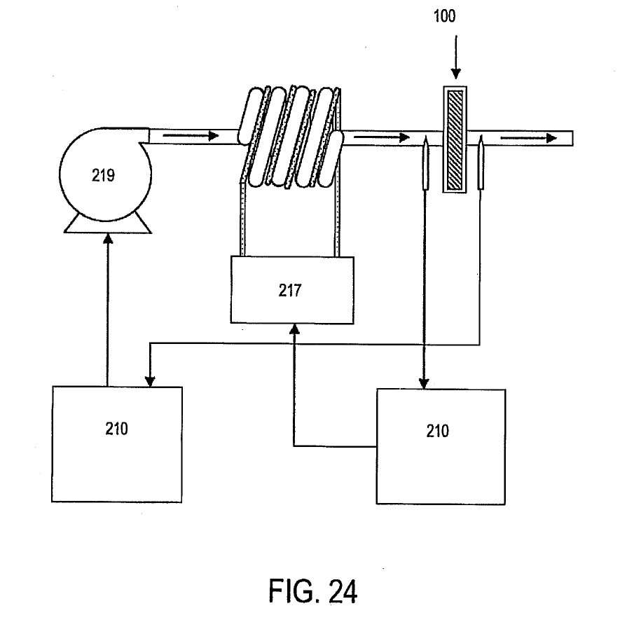

Control System: FIG. 24 shows a schematic block diagram of the control subsystems of FIG. 22 controlling the various components of FIG. 21. In this control situation, there are two process variables which can be controlled and two which can be manipulated. There are a number of options for a control strategy.

In a multiloop control strategy, each manipulated variable depends on only a single controlled variable, i.e., a set of conventional feedback controllers. This strategy may consist of using n standard FB controllers (e.g., PID), one for each controlled variable. The steps may be as follows: (1) Select controlled and manipulated variables; (2) Select pairing of controlled and manipulated variables; (3) Specify types of FB controllers. Example: 2×2 system.

The only variable that can affect the amount of temperature drop is air flow. For this reason the temperature sensor after the CPP will control the air pump and the temp sensor before the CPP will control the Induction heater (after initial heat up).

Thus, in an aspect, there is provided a vaporizer apparatus, comprising: a holder for holding a compressed tablet formed from a plant source material; a microprocessor; a controlled air flow; and a controlled heat source; wherein the microprocessor is adapted to control the air flow and the heat source to vaporize the compressed tablet received in the holder at a desired rate.

In an embodiment, the vaporizer apparatus is adapted to recognize a type of compressed tablet placed into the holder based on one or more distinguishing features.

In another embodiment, the vaporizer apparatus is adapted to recognize the type of compressed tablet placed into the holder based on the shape of the compressed tablet.

In another embodiment, the vaporizer apparatus is adapted to recognize the type of compressed tablet placed into the holder based on a pattern of features formed into the compressed tablet.

In still another embodiment, the pattern of features formed into the compressed tablet comprises holes or ribbed edges formed into the compressed tablet and detectable by sensors.

In another embodiment, the vaporizer apparatus is adapted to recognize the type of compressed tablet placed into the holder based on a machine readable label.

In another embodiment, the machine readable label is one or more of a bar code, a QR code, or an RFID tag provided on the compressed tablet.

In another embodiment, the machine readable label is one or more of a bar code, a QR code, or an RFID tag provided on packaging for one or more compressed tablets.

In yet another embodiment, the microprocessor is adapted to control the heat source to set a temperature profile over a period of time based on the recognized type of compressed tablet placed into the holder.

In another embodiment, the microprocessor is further adapted to control the heat source to set a temperature profile over a period of time based on selected therapeutic compounds desired to be released from the recognized type of compressed tablet placed into the holder.

In another embodiment, the microprocessor is adapted to control the air flow over a period of time based on a desired dosage of selected therapeutic compounds desired to be delivered for inhalation.

In another embodiment, the microprocessor is adapted to receive one or more signals from one or more precision temperature sensors for monitoring the temperature of the air flow.

In another embodiment, the microprocessor is adapted to control the air flow by adjusting a speed of an air pump creating the air flow.

In another embodiment, the microprocessor is adapted to receive one or more signals from one or more precision flow sensors for monitoring the air flow.

In another embodiment, the vaporizer apparatus further comprises a carousel adapted to receive a disc cartridge packaging a plurality of compressed tablets.

In another embodiment, the vaporizer apparatus further comprises perforators for perforating the disc cartridge to prepare one of the plurality of compressed tablets packaged in the disc cartridge for heating by the controlled heat source.

In another embodiment, the microprocessor is adapted to control a motor for rotating the carousel in order to position a first one of the plurality of compressed tablets packaged in the disc cartridge for heating by the controlled heat source.

In another embodiment, the microprocessor is further adapted to advance the carousel to position another a second one of the one of the plurality of compressed tablets packaged in the disc cartridge for heating by the controlled heat source when the first one of the plurality of compressed tablets is spent.

In another embodiment, the holder is further adapted to also receive a mesh container or basket for holding a prepared and measured amount of loose fill plant source material.

In another embodiment, the microprocessor is further adapted to recognize a type of loose fill plant source material placed into the holder based on a machine readable label provided on the container or basket.

In another embodiment, the microprocessor is adapted to control the heat source to set a temperature profile over a period of time based on the recognized type of loose fill plant source material placed into the holder.

In another embodiment, the microprocessor is adapted to control the air flow over a period of time based on a desired dosage of therapeutic compounds desired to be delivered for inhalation.

While illustrative embodiments of the invention have been described above, it will be appreciate that various changes and modifications may be made without departing from the scope of the present invention. For example, while the tablet has been shown as a relatively flat, wide cylinder, it will be appreciated that this shape is not limiting. Alternatively, the tablet may be an elongated cylindrical shape which may obviate the need for through holes by increasing the surface area relative to the mass of the tablet.

1. A vaporizer apparatus, comprising:
a carousel holder adapted to receive a disc cartridge packaging a plurality of compressed tablets formed from a plant source material;
a microprocessor;
a controlled air flow; and
a controlled heat source;
wherein the microprocessor is adapted to:
recognize a type of the compressed tablet placed into the carousel holder based on one or more machine identifiable codes on the packaging;
prepare one of the plurality of compressed tablets packaged within the disc cartridge for heating by the controlled heat source;
and
control the air flow, the heat source, and operation of the carousel based on the recognized type of compressed tablet to vaporize the compressed tablet received in the carousel holder in turn within the disk cartridge at a desired rate.

2. The vaporizer apparatus of claim 1, wherein the vaporizer apparatus is adapted to recognize a type of compressed tablet placed into the holder based on one or more distinguishing features.

3. The vaporizer apparatus of claim 2, wherein the vaporizer apparatus is adapted to recognize the type of compressed tablet placed into the holder based on the shape of the compressed tablet.

4. The vaporizer apparatus of claim 2, wherein the vaporizer apparatus is adapted to recognize the type of compressed tablet placed into the holder based on a pattern of features formed into the compressed tablet.

5. The vaporizer apparatus of claim 1, wherein the pattern of features formed into the compressed tablet comprises holes or ribbed edges formed into the compressed tablet and detectable by sensors.

6. The vaporizer apparatus of claim 2, wherein the vaporizer apparatus is adapted to recognize the type of compressed tablet placed into the holder based on a machine readable label.

7. The vaporizer apparatus of claim 6, wherein the machine readable label is one or more of a bar code, a QR code, or an RFID tag provided on the compressed tablet.

8. The vaporizer apparatus of claim 6, wherein the machine readable label is one or more of a bar code, a QR code, or an RFID tag provided on packaging for one or more compressed tablets.

9. The vaporizer apparatus of claim 2, wherein the microprocessor is adapted to control the heat source to set a temperature profile over a period of time based on the recognized type of compressed tablet placed into the holder.

10. The vaporizer apparatus of claim 9, wherein the microprocessor is further adapted to control the heat source to set a temperature profile over a period of time based on selected therapeutic compounds desired to be released from the recognized type of compressed tablet placed into the holder.

11. The vaporizer apparatus of claim 2, wherein the microprocessor is adapted to control the air flow over a period of time based on a desired dosage of selected therapeutic compounds desired to be delivered for inhalation.

12. The vaporizer apparatus of claim 11, wherein the microprocessor is adapted to receive one or more signals from one or more precision temperature sensors for monitoring the temperature of the air flow.

13. The vaporizer apparatus of claim 11, wherein the microprocessor is adapted to control the air flow by adjusting a speed of an air pump creating the air flow.

14. The vaporizer apparatus of claim 11, wherein the microprocessor is adapted to receive one or more signals from one or more precision flow sensors for monitoring the air flow.

15. The vaporizer apparatus of claim 1, further comprising perforators for perforating the disc cartridge to prepare one of the plurality of compressed tablets packaged in the disc cartridge for heating by the controlled heat source.

16. The vaporizer apparatus of claim 1, wherein the microprocessor is adapted to control a motor for rotating the carousel in order to position a first one of the plurality of compressed tablets packaged in the disc cartridge for heating by the controlled heat source.

17. The vaporizer apparatus of claim 16, wherein the microprocessor is further adapted to advance the carousel to position another a second one of the one of the plurality of compressed tablets packaged in the disc cartridge for heating by the controlled heat source when the first one of the plurality of compressed tablets is spent.

18. The vaporizer apparatus of claim 1, wherein the holder is further adapted to also receive a mesh container or basket for holding a prepared and measured amount of loose fill plant source material.

19. The vaporizer apparatus of claim 18, wherein the microprocessor is further adapted to recognize a type of loose fill plant source material placed into the holder based on a machine readable label provided on the container or basket.

20. The vaporizer apparatus of claim 19, wherein the microprocessor is adapted to control the heat source to set a temperature profile over a period of time based on the recognized type of loose fill plant source material placed into the holder.

21. The vaporizer apparatus of claim 19, wherein the microprocessor is adapted to control the air flow over a period of time based on a desired dosage of therapeutic compounds desired to be delivered for inhalation.
